(19) United States
(12) Patent Application Publication
(10) Pub. No.: US 2018/0263266 A1
(43) Pub. Date: Sep. 20, 2018

(54) AN APPARATUS FOR ULTRA-FINE GRINDING OF RED GINSENG, AND A METHOD FOR PRODUCING WHOLE RED GINSENG EXTRACT AND LIQUID WITH MAXIMIZED NUTRITION ABSORPTIVITY BY ENZYME FERMENTATION

(71) Applicant: HEALTH BALANCE CO., LTD., Gyeryong-si (KR)

(72) Inventors: Jum Kyun LEE, Seoul (KR); Heing Ki MIN, Seoul (KR)

(21) Appl. No.: 15/517,559

(22) PCT Filed: Jul. 11, 2016
(86) PCT No.: PCT/KR2016/007478

§ 371 Date: Apr. 7, 2017

(30) Foreign Application Priority Data
Jan. 6, 2016 (KR) 10-2016-0001510
Jan. 6, 2016 (KR) 10-2016-0001513
Jan. 6, 2016 (KR) 10-2016-0001515
Jan. 6, 2016 (KR) 10-2016-0001518

(51) Int. Cl. A23L 5/30; A23L 5/20; A23L 33/105; C12P 1/04; B02C 4/30; B02C 4/42; B02C 19/18; B02C 23/08

(52) U.S. Cl. CPC A23L 5/30; C12Y 302/01021; A23L 33/105; C12P 1/04; B02C 4/30; B02C 4/42; B02C 19/186; B02C 23/08; A23V 2002/00; A23V 2250/2124; A23V 2300/10; A23V 2300/24; A23V 2300/26; A23V 2300/31; A23V 2300/38; A23V 2300/48; A23Y 2220/00; A23Y 2300/00; A23L 5/25

(57) ABSTRACT

This application describes a method for preparing a fermented red ginseng powder dispersion with improved dispersibility and ginsenoside content. The method includes (a) grinding red ginseng to obtain powdered red ginseng; (b) introducing the powdered red ginseng into a mixed liquid including β-Glucosidase to react with the powdered red ginseng; (c) inoculating the mixed liquid with a fermented strain for fermentation; and (d) controlling a concentration of the mixed liquid to a predetermined range.

### CROSS-REFERENCE TO RELATED APPLICATIONS

This application is the National Stage Entry of International Patent Application No. PCT/KR2016/007478, filed on Jul. 11, 2016, and claims priority from and the benefit of Korean Patent Application No. 10-2016-0001513, filed on Jan. 6, 2016, Korean Patent Application No. 10-2016-0001510, filed on Jan. 6, 2016, Korean Patent Application No. 10-2016-0001515, filed on Jan. 6, 2016, and Korean Patent Application No. 10-2016-0001518, filed on Jan. 6, 2016, each of which is incorporated by reference for all purposes as if fully set forth herein.

### BACKGROUND

### Field

The present invention relates to a method for preparing fermented red ginseng powder dispersion with improved dispersibility and ginsenoside content.

### Discussion Of The Background

According to plant taxonomy, ginseng is a perennial shade-loving herbaceous plant belonging to Panax . of Araliaceae, and it has been used as an important oriental medicine for a long time. Saponin contained in ginseng has physiological activities such as anti-cancer activity, anti-oxidation activity, prevention of arteriosclerosis and hypertension, liver function improvement, anti-fatigue activity, anti-stress activity, prevention of aging, promotion of brain activity, anti-inflammatory activity, treatment of allergic diseases, promotion of protein synthesis ability, etc. Additionally, Korean red ginseng is known for having excellent physiological activities such as anti-oxidation activity, antihypertensive activity, improvement of alcoholic hyperlipidemia, antidiabetic activity, etc.

Ginseng is generally classified into white ginseng and red ginseng according to processing methods. White ginseng refers to ginseng which was dug out from the field without being processed, that is, dried fresh ginseng. Red ginseng is made by steaming, drying and processing fresh ginseng, which accompanies various chemical changes such as saponin modification, change in amino acid, etc. during the preparation process.

Red ginseng produces saponin ingredients such as ginsenoside Rg2, Rg3, Rh1, Rh2, etc., which do not exist in ginseng, by the heat applied during the preparation process. The unique active ingredients of red ginseng exhibit excellence in the prevention of cancer, inhibition of cancer cell growth, antihypertensive activity, protection of brain nerve cell, improvement of learning ability, antithrombotic activity, antioxidation activity, etc., and thus remarkable pharmacological effects could be expected.

Meanwhile, recently, as the consumers are greatly interested in the effects of red ginseng on health promotion, the development of relevant products is on the rise.

Among the red ginseng products, the most representative product is a product extracted by means of water or another solvent with ginseng as a raw material. The active ingredients of red ginseng are dissolved in the extracted product and are processed to have an easily-fed shape. However, even if the active ingredients in red ginseng are extracted through the extraction process, since part of the active ingredients is not extracted and left in red ginseng, a great amount of ingredients including ginsenoside useful for human body are not absorbed, but are left out.

In general, for example, a liquid type red ginseng extraction product including the active ingredients of red ginseng is prepared by mixing red ginseng extracts and other raw materials in purified water, or by preparing red ginseng extracts and manufacturing them right away. However, the red ginseng products include low ginsenoside contents or could not keep the flavor of red ginseng, which leads to insufficiency in functionality and product efficiency.

Accordingly, various methods for increasing extraction efficiency were considered. However, there was a limitation on completely extracting the active ingredients in the red ginseng. Additionally, since active ingredients are inevitably left in the red ginseng used as the raw material for extraction, the methods are not satisfactory in terms of cost or effect.

Recently, in order to solve the above-mentioned problems, products in the form of powder dispersion has been developed, so that an entire red ginseng can be taken in completely. The red ginseng powder dispersion is prepared by grinding the entire red ginseng into fine powder and suspending red ginseng particles in the liquid. Since the entire red ginseng can be taken in, the effects therefrom will be remarkable. Additionally, since the red ginseng powder dispersion is in a form to be easily fed, its product efficiency will also be excellent.

However, in the red ginseng powder dispersion, since the red ginseng particles are suspended in the liquid, the particles may be precipitated or aggregated as time goes by, thereby deteriorating the texture of food or sense of taste. Especially, when granulating the particles to improve the texture of food and dispersibility, the aggregation may rather increase.

Accordingly, various additives such as emulsifiers, etc. were introduced in order to improve dispersibility of the red ginseng powder dispersion. However, due to the consumer's negative perception on the additives, there was a limitation on reducing product efficiency or effectively dispersing red ginseng particles. Thus, various attempts to improve functionality in addition to dispersibility of red ginseng powder dispersion products have been made.

Meanwhile, even though red ginseng or ginseng includes a great amount of major saponin (primary products) such as ginsenoside Rb1, Rb2, Rc, Rd, Re, etc., the major saponin is not directly absorbed into human bodies, but a part thereof is absorbed after being dissolved by an intestinal bacteria flora or an enzyme inside the body, and then converted into minor saponin (secondary metabolite) such as ginsenoside F1, F2, Rg3, compound-K, etc., to express its efficacy. Additionally, even though red ginseng has a remarkable pharmacological activity, the actual pharmacological effect of red ginseng may differ depending on the individual due to the difference in distribution and activation of intestinal microorganism per individual.

Accordingly, in order to solve the above-mentioned problem, research and development are actively conducted on the preparation of fermented red ginseng which converts or increases red ginseng into an ingredient of a final metabolite through a fermentation process using intestinal microorganism.

### SUMMARY

The present invention is to solve the above problems, and it aims to provide a method for preparing red ginseng powder dispersion with improved dispersibility of red ginseng particles.

Additionally, the present invention is to provide a method for preparing red ginseng powder dispersion with increased absorption ratio of useful ingredients included in red ginseng.

According to one aspect of the present invention, the present invention provides a method for preparing fermented red ginseng powder dispersion which includes the steps of (a) grinding red ginseng to obtain powered red ginseng; (b) introducing the powdered red ginseng into a mixed liquid including β-Glucosidase to react with the powdered red ginseng; (c) inoculating the mixed liquid with a fermented strain for fermentation; and (d) controlling a concentration of the mixed liquid to a predetermined range.

According to one embodiment, the present invention may further include the steps of (e) homogenizing with a homogenizer; and (f) performing a low temperature concentration under a vacuum state of 40° C. or below.

According to one embodiment, the powered red ginseng in step (a) may have a particle size of 2 to 20 μm.

According to one embodiment, the grinding in step (a) may be performed under a temperature of −20 to −5° C.

According to one embodiment, the powered red ginseng in step (a) may have a particle size of 2 to 20 μm.

According to one embodiment, step (b) may be performed under a pressure of 1.5 to 4 bar for 1 to 5 hours.

According to one embodiment, step (b) may be performed at a temperature of 30 to 45° C.

According to one embodiment, the mixed liquid in step (b) may include red ginseng extracts.

According to one embodiment, the red ginseng extracts may be prepared with desalted deep ocean water as a solvent.

According to one embodiment, the fermented strain may be at least one strain selected from a group consisting of Monascus sp., Lactobacillus sp., Bifidobacterium sp., Prevotella sp., Fusobacterium sp., and Eubacterium sp.

According to one embodiment, the strain of Monascus sp. may be at least one strain selected from a group consisting of Monascus anka, Monascus purpureus, Monascus pilosus, Monascus ruber, Monascus kaoliang, and Monascus kaling.

According to one embodiment, step (c) may be performed at a temperature of 25 to 35° C. for 3 to 15 days.

According to one embodiment, the present invention may further include the step of performing a heat treatment on the red ginseng before step (a).

According to one embodiment, the heat treatment may supply moisture to a surface of the red ginseng and radiate far infrared rays at the red ginseng.

According to one embodiment, the heat treatment may be performed for 1 to 4 hours.

According to one embodiment, step (e) may be performed under a pressure of 200 to 800 bar for more than 3 times.

According to one embodiment, step (f) may be performed under a vacuum state of a pressure of 1 to 20 kPa.

According to another aspect of the present invention, the present invention provides a method for preparing red ginseng nanopowder which includes the steps of (a) coarsely grinding red ginseng into a particle size of 90 to 150 μm; (b) radiating far infrared rays at the red ginseng; (c) finely grinding the red ginseng into a particle size of 2 to 20 μm; (d) adding β-Glucosidase to the finely ground red ginseng for reaction; and (e) inoculating a fermented strain for fermentation.

According to one embodiment, step (a) may be performed by a low speed rotation at 100 to 500 rpm.

According to one embodiment, the far infrared rays in step (b) may have a wavelength of 20 to 40 μm.

According to one embodiment, step (c) may further include forming a vortex and inducing a collision among particles of red ginseng to finely grind the red ginseng.

According to one embodiment, step (c) may further include radiating ultrasonic waves to increase a collision frequency among the particles of red ginseng.

According to one embodiment, the ultrasonic waves in step (c) may have a vibration frequency of 15 to 20 KHz and an amplitude of 5 to 50 μm.

According to one embodiment, step (c) may be performed at a temperature of −20 to −5° C.

According to still another aspect of the present invention, the present invention provides an apparatus for preparing red ginseng nanopowder including a first grinding part including at least one spiral roller configured to rotate; a heat treating part configured to radiate far infrared rays; a second grinding part configured to form a vortex and configured to introduce collision among particles of red ginseng to finely grind the red ginseng; and a separating part configured to capture the particles of red ginseng discharged from the second grinding part.

According to one embodiment the spiral roller may be configured to rotate at a speed of 100 to 500 rpm.

According to one embodiment, the heat treating part may be configured to radiate far infrared rays with a wavelength of 20 to 40 μm.

According to one embodiment, the second grinding part may be configured to increase the collision frequency among the particles of red ginseng by radiating ultrasonic waves.

According to one embodiment, the ultrasonic waves may have a vibration frequency of 15 to 20 KHz and an amplitude of 5 to 50 μm.

According to one embodiment, the second grinding part may include at least one cooling apparatus configured to supply a low temperature air.

According to one embodiment, the low temperature air may have a temperature of −20 to −5° C.

According to one embodiment, the separating part may include a vacuum pump configured to decrease an inner pressure.

The red ginseng powder dispersion prepared according to the present invention increases hydrophilicity of a particle surface of red ginseng by fermentation so that the dispersion may be stably dispersed in a liquid, and aggregation and precipitation among the particles may be minimized with the passage of time.

The red ginseng powder dispersion prepared according to the present invention remarkably increases the content of ginsenoside which is in a form to be easily absorbed into the body by fermentation, and thus may have excellent functionality as well as taste and flavor, thereby satisfying the preference of consumers as a high-quality product.

The effect of the present invention is not limited to the above effects, and should be construed to include all effects that may be inferred from the features set forth in the detailed description or the claims.

### BRIEF DESCRIPTION OF THE DRAWINGS

FIG. 1 is a diagram illustrating a method for preparing fermented red ginseng powder dispersion according to one embodiment of the present invention;

FIG. 2 is a diagram illustrating a method for preparing fermented red ginseng powder dispersion according to one embodiment of the present invention;

FIG. 3 is a diagram illustrating a method for preparing red ginseng nanopowder according to one embodiment of the present invention; and

FIG. 4 is a diagram illustrating an apparatus for preparing red ginseng nanopowder according to one embodiment of the present invention.

### DETAILED DESCRIPTION OF THE ILLUSTRATED EMBODIMENTS

Terms used in the present specification are general terms which are currently widely used in consideration of function of the present invention. However, the terms may differ depending on the intention of a person skilled in the art or precedents, the appearance of new technology, etc. Additionally, there are terms which the applicant optionally designates, and in this case, the meanings thereof will be described in detail in the detailed description of the invention. Thus, the terms used in the present invention are not simply defined as the names of terms, but should be defined based on the meanings the terms have and the whole contents of the present invention.

Unless defined to the contrary, all terms including technical or scientific terms used herein have the same meanings as those generally understood by a skilled person in the art. The terms defined in the dictionary which are generally used should be interpreted to have the same meaning as the meaning of relevant technology in context. Additionally, unless the present invention clearly defines, the terms are not interpreted idealistically or excessively formally.

A numerical range includes numerical values defined in the above range. All maximum numerical limitation given throughout the present specification includes all lower numerical limitations as clearly described. All minimum numerical limitations given throughout the present specification include all higher numerical limitations clearly described. All numerical limitations given throughout the present specification include all numerical limitations within a broader numerical range as clearly described.

Hereinafter, embodiments of the present invention will be described in detail, but it is obvious that the present invention is not limited thereto.

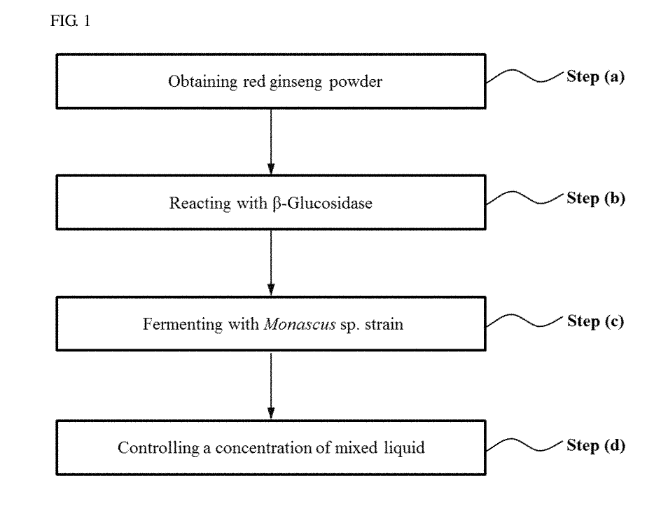

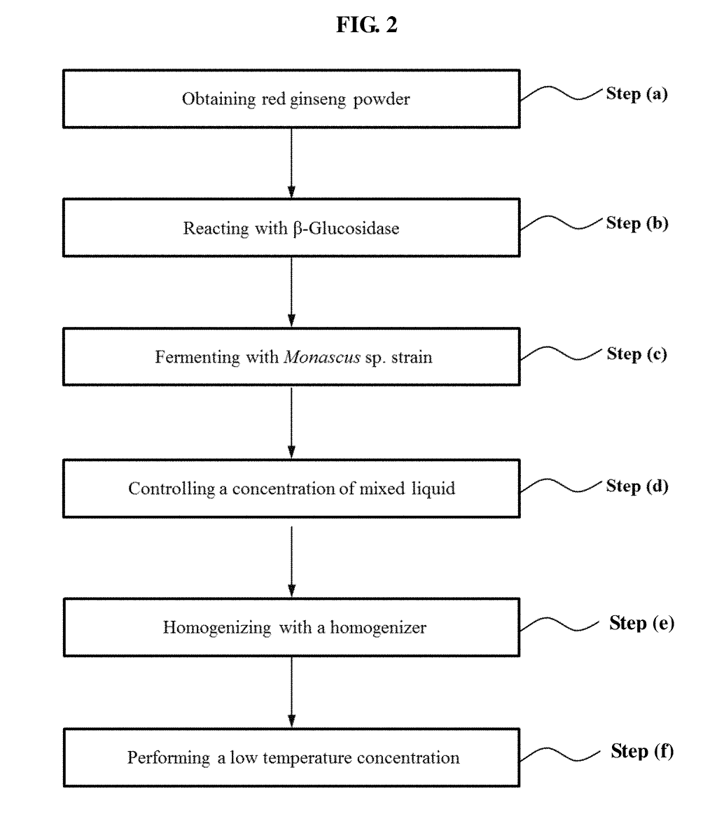

FIGS. 1 and 2 are diagrams illustrating a method for preparing fermented red ginseng powder dispersion according to one embodiment of the present invention.

Referring to FIGS. 1 and 2, the method for preparing fermented red ginseng powder dispersion may include the steps of (a) grinding red ginseng to obtain powder; (b) introducing the powder into a mixed liquid including β-Glucosidase to react the powder; (c) inoculating the mixed liquid with fermented strain for fermentation; and (d) controlling a concentration of the mixed liquid to a predetermined range.

The “red ginseng” refers to ginseng which becomes red by steaming and drying fresh ginseng, and the origin, type and shape, etc. of ginseng are not limited. For example, the ginseng may be Panax ginseng C. A. Meyer, Panax quinquefolium, Panax notoginseng, Panax japonicum, Panax trifolium or Panax pseudoginseng which are commonly used, and all parts of the ginseng may be used.

In other words, the ginseng refers to any ginseng generally used for the preparation of red ginseng, and types thereof are not particularly limited. For example, fresh ginseng, white ginseng, mountain cultivated ginseng, etc. may be used as the ginseng, and the types thereof are not limited. All parts of the ginseng may be used. For example, the ginseng may be whole ginseng or ginseng radix palva, and the whole ginseng and ginseng radix palva may be used together.

Meanwhile, in the method for preparing for the powder dispersion, as raw materials of red ginseng, red ginseng prepared by steaming fresh ginseng or fresh ginseng which is not processed in consideration of quality of final product or demand of the market may be directly used.

Meanwhile, the grinding in the step (a) may be performed at a temperature of −20 to −5° C., and a particle size of the powder may be 2 to 20 μm.

The grinding is performed at a low temperature which prevents ingredients useful for human body from being damaged by the frictional heat. Additionally, water solubility index (WSI) may increase by granulating the powder. Especially, the red ginseng particles are ground at a low temperature, so an aggregation phenomenon among the particles during the ultrafine grinding process may be minimized.

Additionally, the powder has ultrafine particles, thereby reducing water absorption index (WAI) and swelling power (SW). However, since water solubility increases, the ability to combine with water may increase. Additionally, the powder may be uniformly dispersed in water without causing cohesion or precipitation among the fine particles. The ultrafine powder has a great specific surface area representing a surface area per unit weight, and the great specific surface area increases the contact area between a solid and a solvent, thereby increasing solubility of a material with low solubility inside the body fluid. Thus, since the ultrafine powder may be dissolved in the human body faster than the coarsely ground powder, the absorption of red ginseng ingredients may increase, thereby improving bioavailability of red ginseng ingredients.

According to one embodiment, in the step (b), the powder may be introduced into the mixed liquid including β-Glucosidase for reaction. The step (b) reacts red ginseng in a mixed liquid including an enzyme converting ginsenoside for a predetermined period of time before the step of fermenting microorganism so that the enzyme may be absorbed in the red ginseng.

The “β-Glucosidase” is one type of cellulase which is an enzyme dissolving cellulose. The β-Glucosidase may convert cellobiose which is a glucose dimer into glucose, and effectively dissolve insoluble cellulose. Additionally, the β-Glucosidase may be derived from aspergillus niger, but is not particularly limited thereto in case of having an equivalent conversion activity. The β-Glucosidase is excellent in saponin dissolving power, and may convert major saponin such as Rb1, Rb2, Rc, Rd, Re, etc. into minor metabolite. That is, the β-Glucosidase may convert protopanaxadiol (PPD)-type or protopanaxatriol (PPT)-type major saponin into soluble minor saponin.

The “protopanaxadiol (PPD)-type saponin” is a dammarane-based saponin, which refers to a ginsenoside having two hydroxyl groups (—OH) attached to aglycone, for example, Rb1, Rb2, Rc, Rd, Re, Rf or Rg1. Additionally, the PPD-type saponin may include all saponins which may be converted into ginsenoside Rg3 by the activity of the ginsenoside glycosidase. Also, the soluble “minor saponin” means a minor saponin relatively easily absorbed in the body, which is produced by sequentially hydrolyzing the glucose of the 20th carbon of the PPD-type or PPT-type saponin, which is not easily absorbed in the body, but the types thereof are not particularly limited. The minor saponin may include ginsenoside Rd, Rg3, Rg2, Rh1, Rh2, F1, C—O, or C-Mc1, and preferably may be Rd, Rg3, Rg2 or Rh1.

The β-Glucosidase enzyme may promote the conversion of major saponin ingredients in the red ginseng in the following fermentation step, and increase the ratio of the hydrophilic group of a particle surface, and thus the β-Glucosidase enzyme may increase the absorption ratio of useful ingredients in the body and dispersibility of ground red ginseng particle. Especially, due to the acidic properties of intestinal β-Glucosidase enzyme, the intestinal β-Glucosidase enzyme may not exert sufficient activity for converting saponin in the red ginseng into useful ingredients. Thus, an enzyme treatment is performed in advance during the process of processing red ginseng product, to improve the functionality of the product.

According to one embodiment, the step (b) may be performed under a pressure of 1.5 to 4 bar for 1 to 5 hours. Since the mixed liquid including the β-Glucosidase may be effectively introduced into the red ginseng under a high pressure, the reaction efficiency may be improved by increasing the pressure to be higher than ordinary conditions. When the pressure is less than 1.5 bar, the absorption efficiency may not increase sufficiently. When the pressure exceeds 4 bar, the absorption ratio may not increase in proportion to the increase of pressure and processing fee would be excessively high. Additionally, when the reaction time is less than 1 hour, the enzyme may not be sufficiently absorbed into the red ginseng. Also, when the reaction time exceeds 5 hours, the absorption efficiency may deteriorate, which may lead to inefficiency in terms of cost and time.

Meanwhile, the step (b) may be performed at a temperature of 30 to 45° C. When the temperature is less than 30° C., the tissue of red ginseng particle may be stiffened, and inner void may be reduced, so introduction efficiency of the enzyme may deteriorate. Additionally, when the temperature exceeds 45° C., the active ingredients vulnerable to heat may be modified or the activity of the enzyme may deteriorate.

In this case, the mixed liquid in the step (b) may be agitated so that the enzyme is effectively introduced into the red ginseng particle, and preferably it may be agitated at 10 to 15 rpm. In general, the agitation may be performed by mechanical apparatuses such as impellers, etc., which are installed in the center of a chamber and rotated by the motor, but it may be freely modified in consideration of the properties of the process system. Meanwhile, when the agitation speed is excessively slow or fast, the desired object may not be achieved, or the active ingredients may be modified or lost. Thus, the agitation speed may be properly controlled to be within the above range.

According to the embodiment, the mixed liquid in the step (b) may further include red ginseng extracts. The “extract” refers to a solvent having the active ingredients included in the extracted raw material transited by contacting the solvent and extracted raw material under a specific condition. The red ginseng extracts may include active ingredients included in the fermented red ginseng.

In general, the fermented red ginseng powder dispersion may be a mixed liquid wherein granulated red ginseng particles are dispersed in purified water. However, in case of replacing the purified water with red ginseng extracts, dispersibility and product efficiency may be further improved.

That is, in the red ginseng extracts, various active ingredients included in the red ginseng are dissolved by the extraction process, so the polarity thereof is relatively high, and thereby the hydrophilic interaction with the granulated red ginseng particles may be improved. Additionally, the red ginseng extracts include a great amount of functional ingredients which are useful for the human body, so remarkable effects for improving health may be implemented.

The type of solvent used in the extraction process is not particularly limited and may vary depending on the processing condition. For example, the red ginseng extracts may be prepared by washing red ginseng raw material with water, drying and grinding the red ginseng raw material, extracting the red ginseng raw material with common methods such as reflux circulation extraction, pressed extraction, ultrasonic-assisted extraction, etc. for a predetermined time with a solvent which weighs 8 and 12 times of the raw material, and filtering the extracted red ginseng. Additionally, the extracts may be obtained in powders by an additional process such as vacuum distillation, or freeze drying, etc.

According to one embodiment, the red ginseng extracts may be prepared with desalted deep ocean water as the solvent.

The deep ocean water means seawater circulating inside the deep sea of 200 m or below, clearly distinguished from surface water which is close to a sea level and greatly affected by precipitation, capacity, vaporization, etc. Seawater continuously circulates without staying in one spot. When the circulating seawater reaches a glacier area, since specific gravity gets higher due to the difference in temperature, water depth becomes 200 m or below. Accordingly, cold seawater may not be mixed with surface water due to the difference in density, and thus a huge layer with a boundary may be made.

The deep ocean water is stable at a low temperature and hardly includes organic matters or pathogenic bacteria, etc. Additionally, the deep ocean water is rich in nutritive salts which are essential for the growth of an oceanophyte, so resource properties such as stability, cleanness, eutrophication, etc. are remarkable. Thus, the red ginseng extracts with the deep ocean water as the solvent contain rich mineral together with active ingredients included in the red ginseng, and thus the effect for improving health may be remarkable, and dispersion stability for the red ginseng particles may increase remarkably.

The desalted deep ocean water may be obtained by applying any technology well-known in the related field. Methods such as flash vaporization method, seawater freezing method, reverse osmosis, ion exchange resin method, electrodialysis method, etc. may be used, but the method is not limited thereto and may be properly selected in consideration of the properties of the process. Specifically, the reverse osmosis refers to a method of removing salinity using a reverse osmosis membrane, and the electrodialysis method refers to a method of selectively reducing the concentration of sodium without changing the concentration of necessary mineral.

Meanwhile, in the step (c), the mixed liquid may be inoculated with fermented strain for fermentation, and the fermentation may be performed for 3 to 15 days at a temperature of 25 to 35° C.

The fermented strain may convert major saponin which has a low absorption ratio for the human body into soluble minor saponin through the ginsenoside metabolic process.

According to one embodiment, the fermented strain may be at least one selected from a group consisting of a strain of Monascus sp., Lactobacillus sp., Bifidobacterium sp., Prevotella sp., Fusobacterium sp., and Eubacterium sp. Preferably, the strain of Monascus sp. may be selected.

The strain of Monascus sp. is a fungus belonging to ascomycetes, which generates a red pigment using starch. Rice which is inoculated with the strain of Monascus sp. and cultivated becomes rice koji referred to as monascus (red rice koji). The strain of Monascus sp. has been widely used for preparing red rice-wine or other various fermented foods, and many reports on the treatment for diseases and medicinal efficacy thereof have been made.

The strain may give hydrophilic properties to the particle surface during the process of converting organic matters of the red ginseng particles. Since the fermented red ginseng particles have increased hydrophilicity, they may be in a stable colloid state by reacting with water. That is, since the hydrophilic colloid may be easily dispersed in water, the fermented red ginseng powder dispersion according to the present invention may have remarkable dispersibility without an additional chemical additive such as an emulsifier, etc. and also have an increased saponin content which is in a form to be easily fed by fermentation, and thereby the effect of promoting health may be improved.

According to one embodiment, the strain of Monascus sp. may be at least one selected from a group consisting of Monascus anka, Monascus purpureus, Monascus pilosus, Monascus ruber, Monascus kaoliang, and Monascus kaling.

The strain of Monascus sp. may have at least one activity selected from a group consisting of esterase, leucine arylamidase, valine arylamidase, stine arylamidase, acid phosphatase, naphtol-AS-phosphohydrolase, α-glucosidase and β-glucosidase. Additionally, the strain of Monascus sp. may convert the protopanaxadiol (PPD)-type or protopanaxatriol (PPT)-type major saponin included in the red ginseng into soluble minor saponin.

Especially, in the step (b), the mixed liquid including the β-Glucosidase reacted with the red ginseng and was converted into a form to be easily fermented. Thus, the fermentation efficiency by the strain of Monascus sp. in the step (c) may be further improved. The β-Glucosidase softens the firm cellulose of the red ginseng, and converts dimers which cannot be easily taken in by the strain of Monascus sp. into monomers. Thus, the culture efficiency of the strain of Monascus sp. may increase remarkably.

According to one embodiment, the step of performing the heat treatment on the red ginseng may be further included before the step (a), and the heat treatment may be performed for 1 to 4 hours.

Since the β-Glucosidase enzyme is introduced into the red ginseng in the step (b), the heat treatment is performed on the red ginseng before the above process so that the mixed liquid may be easily absorbed. As the result of heat treatment, the red ginseng has increased porous properties and flexible fiber tissue. Thus, the absorption ratio of the mixed liquid may increase, and the efficiency of the following fermentation process may be improved by the heat treatment process.

Additionally, the heat treatment may supply moisture to the surface of the red ginseng and radiate far infrared ray. The far infrared ray has a wavelength of 50 to 1,000 μm and is an electromagnetic radiation of an area with a long wavelength among the infrared radiation. Since the far infrared ray has a long wavelength, it may be easily absorbed in the red ginseng, and the red ginseng may be evenly heated as a whole. Especially, the far infrared ray increases the physiological activity of the red ginseng and activates the tissue, so it is suitable for the preparation of high-quality red ginseng products.

In the step (d), the concentration of the mixed liquid may be controlled to a predetermined range. The fermented red ginseng powder dispersion may have different concentrations and viscosities depending on the price of final product and the consumer's demand. Additionally, the ground red ginseng particles and water content may be properly controlled.

In the step of controlling the concentration and viscosity, a thickening agent may be added. The red ginseng powder dispersion has increased solubility of powder particles by the enzyme and fermented strain, and thus it has remarkable dispersibility. However, the quality and properties of the final product may be optimized by adding a small amount of thickening agent depending on the costs or conditions of process.

The thickening agent may be a suspension implementing agent, a precipitation inhibiting agent, a gel forming agent or a bulking agent, but the type thereof is not particularly limited.

According to one embodiment, as shown in FIG. 2, the steps of (e) homogenizing with a homogenizer; and (f) performing a low temperature concentration under a vacuum state of 40° C. or below may be further included after the step (d).

The homogenization means a treatment by a pressurized, ultrasonic or agitated emulsifier. Preferably, an ultrasonic emulsifier or ultrahigh pressurized homogenizer may be used. Especially, the ultrasonic emulsifier may improve the transparency of the powder dispersion, and the ultrahigh pressurized homogenizer has remarkable homogenization. Thus, the time spent for the process may be reduced.

Meanwhile, when the pressure or the number of process is excessively high or low during the homogenization process, the cost or process efficiency may deteriorate. Thus, the processing condition may be properly controlled.

The homogenized dispersion may be concentrated at a low temperature under a vacuum state of 40° C. or below, and preferably under a vacuum state at a pressure of 1 to 20 kPa.

The concentration means an operation of removing moisture and increasing concentration of a solid matter, and the concentration method is not particularly limited. That is, a vaporization concentration method which vaporizes moisture, a freezing concentration method which freezes a solvent and mechanically separates the ice produced, and a reverse osmosis concentration method which separates moisture through a semipermeable membrane by applying a pressure of at least the osmotic pressure of the liquid to be concentrated may be used, but preferably the vaporization concentration method may be used. Especially, since the active ingredients included in the red ginseng extracts may be modified or destroyed at a high temperature, it is preferable to control the temperature at a proper level during the process. Also, the vaporization concentration method which vaporizes the solvent under reduced pressure and concentrates the solvent may be applied.

The red ginseng powder dispersion is concentrated under a vacuum state, so the concentration of the dispersion, that is, a specific gravity of the red ginseng particles could increase at a relatively low temperature, compared to the dispersion medium. However, when setting the temperature to be excessively low, the vaporization of solvent will not be smoothly made, and thus efficiency of entire process could deteriorate. Additionally, when the pressure is less than 1 kPa, the process costs may be excessively high. When the pressure exceeds 20 kPa, the vaporization efficiency may deteriorate. Thus, the pressure and temperature may be properly controlled in consideration of the quality of product and processing condition.

As for the red ginseng powder dispersion, preferably, the content of red ginseng particles is at least 70% by weight as compared to the total dispersion weight in consideration of functionality and product efficiency of the final product. However, the concentration may vary depending on the quality of the final product or the market's demand.

Especially, products in the form of red ginseng extracts which are in a great demand in the market may be implemented through the concentration process, and since the concentration process is performed after the homogenization process, the red ginseng powder may be stably dispersed in the dispersion medium.

Also, in order to control the concentration or viscosity of the red ginseng powder dispersion, thickening agents may be added. The red ginseng powder dispersion has increased solubility of powder particle by the enzyme and fermented strain, and thus it has remarkable dispersibility. However, the quality and properties of the final product may be optimized by adding a small amount of thickening agent depending on the costs or conditions of process.

The thickening agent may be a suspension implementing agent, a precipitation inhibiting agent, a gel forming agent or a bulking agent, but the type thereof is not particularly limited.

According to another aspect of the present invention, the method for preparing red ginseng nanopowder which includes the steps of (a) coarsely grinding red ginseng into a particle size of 90 to 150 μm; (b) radiating far infrared ray; (c) finely grinding the red ginseng into a particle size of 2 to 20 μm; (d) adding β-Glucosidase to the finely ground red ginseng for reaction; and (e) inoculating strain of Monascus sp. for fermentation is provided. Steps (d) and (e) are described above with respect to FIGS. 1 and 2 and will not be repeated for brevity.

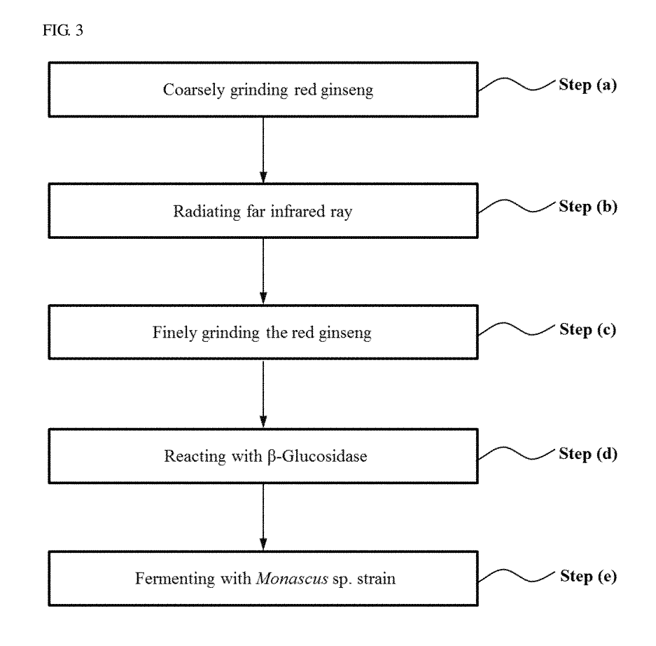

FIG. 3 is a diagram illustrating a method for preparing red ginseng nanopowder according to one embodiment of the present invention.

The method for preparing the red ginseng powders may increase the water solubility index (WSI) by nano-particulating red ginseng, and give hydrophilicity of the surface of red ginseng particles through the fermentation process. Thus, the red ginseng powders prepared by the method may be uniformly dispersed in the dispersion medium and aggregation among the particles may be minimized.

Specifically, the method for preparing the red ginseng nanopowder may sequentially include the steps of coarsely-grinding the red ginseng, radiating far infrared ray, and finely-grinding the red ginseng.

First, in the step (a), the red ginseng may be coarsely ground to a particle size of 90 to 150 μm, and red ginseng powders that are primarily coarsely ground may be finely ground easily.

The coarse grinding means a process of crushing the raw material introduced through a crushing means such as a roller or a milling cutter, etc. into a predetermined size. For example, the crushing means may be a roller, a ball mill, a rod mill, a roller mill, a wheeler mill, a hammer mill, a tumbling mill or a pin mill, but is not limited thereto.

According to one embodiment, the step (a) may be performed by a low speed rotation of 100 to 500 rpm.

Since the coarse grinding is performed at a low speed, the modification of active ingredients by an excessive frictional heat may be inhibited, and contamination or loss of the raw material by the generation of dust may be minimized. Meanwhile, when the rotation speed is less than 100 rpm, the process efficiency deteriorates, and thus an excessive amount of cost and time could be spent for preparation. Additionally, when the rotation speed exceeds 500 rpm, the active ingredients of red ginseng may be lost by the frictional heat.

Meanwhile, far infrared ray may be radiated on the surface of red ginseng in the step (b) after the coarse grinding.

The far infrared ray has a wavelength of 50 to 1,000 μm and is an electromagnetic radiation of an area with a long wavelength among the infrared radiation. Since the far infrared ray increases the physiological activity of the red ginseng and activates the tissue, the effect of the red ginseng powder on the improvement of health may increase. The wavelength of the far infrared ray is not particularly limited, but preferably, optimal conversion properties may be implemented within a range of 20 to 40 μm.

Especially, natural antioxidant materials include high molecules such as polyphenol, tocopherol, flavonoid, etc. which are polymers, and the far infrared ray may change the high molecular polymers to have low molecules, thereby further improving an antioxidant capacity of red ginseng.

The red ginseng may be finely ground to a particle size of 2 to 20 μm in the step (c) after radiating the far infrared ray.

As the red ginseng is ultra-finely ground, water absorption index (WAI) and swelling power (SW) are reduced, but water solubility increases. Thus, the ability to combine with water increases, and the red ginseng may be uniformly dispersed in water without cohesion or precipitation among the fine particles. The ultrafine powders have a great specific surface area representing the surface area per unit weight, and the great specific surface area increases the contact area between the solid and solvent, thereby increasing solubility of a material with low solubility inside the body fluid.

Thus, since the ultrafine powders may be dissolved in the human body faster than the coarsely ground powders, absorption ratio of red ginseng ingredients may increase, thereby improving bioavailability of red ginseng ingredients.

According to one embodiment, the step (c) forms vortex and induces collision among the red ginseng particles, and thus the red ginseng which is coarsely ground in the step (a) may be finely ground. The step (c) introduces the coarsely-ground red ginseng particles with a high speed fluid to form the vortex, and induces the collision among the particles within the vortex to finely grind the red ginseng particles.

In general, a method which is commonly used in the related field may be applied as the method of forming the vortex. For example, the method of blowing compressed air of at least a predetermined atmospheric pressure or water vapor to form vortex, inhaling ground raw material, accelerating it, inducing collision among the particles or collision between the particle and a collision plate to finely grind the particles circulating within the air current may be applied.

Meanwhile, in the step (c), ultrasonic waves are radiated to increase collision frequency among the particles. Since the collision among the particles occurs by the vortex, the particles may be finely ground. However, the grinding efficiency may further increase by radiating ultrasonic waves.

The range of ultrasonic waves is not particularly limited. Preferably, however, the ultrasonic waves may have a vibration frequency of 15 to 20 KHz, and an amplitude of 5 to 50 μm. The ultrasonic waves within the range may give an effective vibration for the circulating red ginseng particles, and increase the collision frequency among the particles and collision strength to improve the fine grinding effect.

In this case, the fine grinding may be performed at a temperature of −20 to −5° C. Since the red ginseng particles circulate within the vortex, they are continuously cooled during the grinding process so an exothermic reaction hardly occurs. However, since the red ginseng particles are finely ground at a very low temperature, the fine grinding process may completely block the modification or loss of active ingredients in the red ginseng by the heat. Also, since the fine grinding process is performed at a very low temperature, the red ginseng particles are frozen and thus their hardness becomes great. Thus, the grinding efficiency by the collision may increase remarkably.

The fermented red ginseng powder dispersion prepared by the above method may be filled in the package material after selectively going through a sterilization method and is then completed.

The sterilization process may be performed by a sterilization process commonly used in the related field. When the sterilization temperature is excessively high, the ginsenoside ingredient of the red ginseng particles may be destroyed or modified, and thus low temperature sterilization may be performed at a temperature of 80° C. or below.

According to another aspect of the present invention, an apparatus for preparing red ginseng nanopowder which includes a first grinding part 101 including at least one spiral roller met for rotation; a heat treating part 102 radiating far infrared ray; a second grinding part 103 forming vortex and inducing collision among particles of red ginseng to finely grind the red ginseng; and a separating part 104 capturing red ginseng particles discharged from the second grinding part 103 is provided.

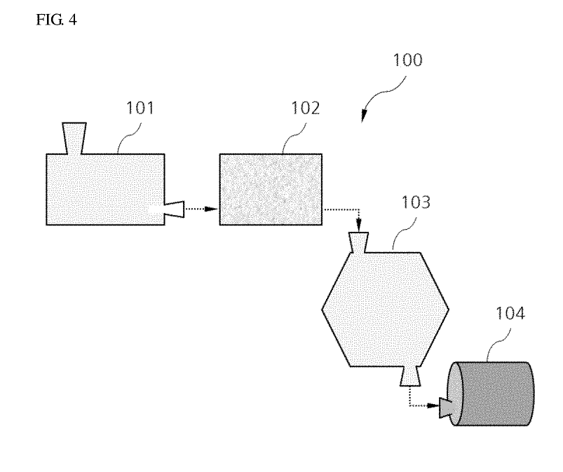

FIG. 4 is a diagram illustrating an apparatus for preparing red ginseng nanopowder according to one embodiment of the present invention. The apparatus for preparing red ginseng nanopowder combines the coarse grinding process with the fine grinding process to effectively nano-particulate the active ingredients of the red ginseng without destroying them.

Referring to FIG. 4, the apparatus for preparing the red ginseng nanopowder may include a first grinding part 101, a heat treating part 102, a second grinding part 103, and a separating part 104. The red ginseng may be finely ground by passing through each constituent of the preparation apparatus, which are sequentially connected.

Specifically, the first grinding part 101 may coarsely grind red ginseng. The coarse grinding grinds the red ginseng to have a particle size of 90 to 150 μm so as to facilitate beforehand processing before performing fine grinding and enable the subsequent fine grinding process. The first grinding part 101 includes a crushing means such as a spiral roller, etc. for coarsely grinding the red ginseng, and may crush the red ginseng or ginseng inserted into a predetermined size.

The first grinding part 101 may grind red ginseng by using a spiral roller, a ball mill, a rod mill, a roller mill, a wheeler mill, a hammer mill, a tumbling mill or pin mill, but is not limited thereto. Additionally, when it is possible to perform coarse grinding so that a beforehand processing may be performed before fine grinding, there is no limitation on the grinding method.

For example, the first grinding part 101 may include a pair of rollers, and the rollers may crush the red ginseng at least two times by controlling a gap to gradually narrow the gap to a different gap. That is, after two pairs of rollers with different gaps are disposed to be adjacent to each other to preliminarily crush the red ginseng, secondarily, the red ginseng may be crushed densely.

In this case, the spiral roller may rotate at a speed of 100 to 500 rpm. When the coarse grinding is performed at a high speed, the active ingredients in the red ginseng may be destroyed by the frictional heat and external force. Accordingly, since the first grinding part 101 performs the coarse grinding at a low speed, the modification of active ingredients by the excessive frictional heat may be minimized, and contamination or loss of the raw material by the generation of dust may be inhibited. Meanwhile, when the rotation speed is less than 100 rpm, the process efficiency deteriorates, and thus an excessive amount of cost and time could be spent for preparation. Additionally, when the rotation speed exceeds 500 rpm, the active ingredients of red ginseng may be lost by the frictional heat.

Meanwhile, the heat treating part 102 radiates far infrared ray to the coarsely-ground red ginseng to increase the physiological activity effect of the red ginseng. Preferably, the far infrared ray with a wavelength of 20 to 40 μm may be radiated.

The heat treating part 102 is connected to the first grinding part 101, and the coarsely-ground red ginseng may be transported by transporting means such as a conveyor belt, etc. The coarsely-ground red ginseng may be transported to a heat treating part 102, stay there for a predetermined time, and be subjected to heat treatment by the far infrared ray, but the method thereof is not limited thereto. For example, the heat treatment method may be applied while being transported by the transporting means.

The heat treating part 102 may include at least one far infrared ray lamp radiating far infrared ray, and the far infrared ray lamp may periodically change a radiation angle by a transmission gear at predetermined intervals to evenly radiate the far infrared ray.

As the far infrared ray has a wavelength of 50 to 1,000 μm and is an electromagnetic radiation of an area with a long wavelength among the infrared radiation, it increases the physiological activity of the red ginseng and activates the tissue, and thus the effect of the red ginseng powders on the improvement of health may increase. The wavelength of the far infrared ray is not particularly limited. Preferably, however, the optimal conversion properties may be implemented at a range of 20 to 40 μm.

Meanwhile, the second grinding part 103 forms the vortex and induces the collision among the particles of red ginseng to finely grind the red ginseng. The red ginseng has ultrafine particles, thereby reducing water absorption index (WAI) and swelling power (SW). However, since water solubility increases, the ability to combine with water may increase. Additionally, the powder may be uniformly dispersed in water without causing cohesion or precipitation among the fine particles. The ultrafine powder has a great specific surface area representing a surface area per unit weight, and the great specific surface area increases the contact area between a solid and a solvent, thereby increasing solubility of a material with low solubility inside the body fluid.

The second grinding part 103 introduces the coarsely-ground red ginseng particle with a high speed fluid to form the vortex, and induces the collision among the particles in the vortex to finely grind the particles of the red ginseng.

In general, a method which is commonly used in the related field may be applied as the method of forming the vortex. For example, the particles circulating within the air current may be finely ground by blowing compressed air of at least a predetermined atmospheric pressure or water vapor from a specific nozzle to form vortex, and inducing collision among the particles or collision between the particle and a collision plate.

The second grinding part 103 has a structure that may form a vortex in a commonly known chamber, and it may include a cylindrical chamber and an injection nozzle injecting high pressure gas. The red ginseng particles introduced together with the fluid rotate according to the flow of the fluid, and the finely ground red ginseng particles may be discharged to the separating part 104 by a centrifugal force. In this case, inert gas such as helium, etc. may be injected to form the vortex, or air safe from oxidization may be used, but the type thereof is not particularly limited.

The apparatus for forming the vortex may be used regardless of the type or structure thereof. Preferably, the vortex may be formed at a rotation speed of 3,000 to 5,000 rpm. Additionally, the fine grinding by the vortex may be performed under the conditions causing a decrease of at least 20% in volume mean diameter, and a decrease of up to 80% in number-average diameter.

Meanwhile, the second grinding part 103 may increase collision frequency among the particles of red ginseng by radiating ultrasonic waves. That is, the second grinding part 103 forms the vortex and induces collision among the particles to finely grind the particles of red ginseng. However, when radiating the ultrasonic waves during the fine grinding process, the collision among the particles is frequently made and collision strength becomes great, and thus the grinding efficiency may increase.

In this case, the ultrasonic waves may have a vibration frequency of 15 to 20 KHz, and an amplitude of 5 to 50 μm. The ultrasonic waves within the above ranges may give an effective vibration on the particles of circulating red ginseng and increase collision frequency among the particles and collision strength, thereby improving the fine grinding effect.

Additionally, the second grinding part 103 may include at least one cooling apparatus supplying low temperature air, and the low temperature of the air may be a temperature of −20 to −5° C. The fine grinding process is performed at a very low temperature, so the modification or loss of the active ingredients in the red ginseng by the heat may be blocked. Additionally, since the fine grinding process is performed at a very low temperature, the particles of the red ginseng become hard, and thus the grinding efficiency by collision may increase remarkably.

According to one embodiment, the second grinding part 103 may include a heat exchange core for cooling the air and a nozzle for injecting the cooled low temperature air to the inside, and various cooling methods used in the related field may be applied.

Meanwhile, the separating part 104 may capture the particles of red ginseng discharged from the second grinding part 103. The separating part 104 is connected to one end of the second grinding part 103, and the second grinding part 103 may selectively separate the fine particles of red ginseng.

In the chamber of the second grinding part 103 where the fine grinding process is performed, the red ginseng particles which are sufficiently ground and not sufficiently ground may be mixed. Additionally, the separating part 104 connected to the second grinding part 103 may select only the red ginseng particles ground to have a predetermined particle size or less, discharge them outside and classify them.

The classification means an operation of separating materials with different sizes or properties, and the separating part 104 may select the red ginseng particles with a predetermined particle size or less from the powder where particles with various sizes are mixed, and allow the particles to pass.

According to one embodiment, the separating part 104 prevents powder with a great particle size of at least a predetermined particle size from being introduced into the separating part 104, and selectively allows only particles with a predetermined size or below to pass by using the centrifugal force generated in a plurality of blades installed in a centrifugal rotor.

Additionally, in order to increase the separation efficiency of the finely ground particles of red ginseng, the separating part 104 may form a low pressure atmosphere, and include a vacuum pump decreasing the pressure inside the separating part 104 so that the finely ground particles of red ginseng may be effectively classified according to the particle size.

The low pressure atmosphere by the vacuum pump may form an ascending air current, and the red ginseng particles circulating in the ascending air current may be finely classified by the pressure action of the vacuum pump.

Hereinafter, the present invention will be described with reference to the embodiments, but it is obvious that the present invention is not limited thereto.

### PREPARATION EXAMPLE 1

### Seed Culture of M. Purpureus (MP)

The M. purpureus (MP) used in the solid culture was distributed from the RDA-Genebank center, National Institute of Agricultural Science of Rural Development Administration (RDA) (Suwon, Korea). The purpureus was cultured in a potato dextrose agar (PDA, Difco, Sparks, Md., USA) plate medium at 25° C. for about 10 days, inoculated in an erlenmeyer flask containing potato dextrose broth (PDB, Difco), and sub-cultured in a shaking incubator (Jeio tech, Daejeon, Korea) about every 5 days. The M. purpureus used a seed culture sub-cultured for 3 times in the PDB medium.

### PREPARATION EXAMPLE 2

### Separation of β-Glucosidase

Aspergillus niger strain obtained from the RDA-Genebank center of Rural Development Administration (RDA) was cultured in a potato dextrose broth (PDB) liquid medium at 30° C. for 3 days. The culture medium was centrifuged at 7,000 rpm for 10 minutes, the supernatant and precipitate were separated, and the supernatant was removed. The precipitate was suspended with 0.1M potassium phosphate buffer, and the suspension was crushed with ultrasonic waves. The crushed product was centrifuged at 12,000 rpm for 5 minutes to separate the supernatant from the precipitate, and the supernatant including an enzyme was obtained. By performing freeze drying, β-Glucosidase enzyme was separated from the aspergillus niger strain.

### EXAMPLE 1

After purchasing 6 year-old fresh ginseng harvested in 2008 in Jeungpyeong of Chungbuk, red ginseng was prepared by steaming the ginseng. The prepared red ginseng was stored at 10° C. for experiment.

The prepared red ginseng was ground at a particle size of 10 μm using a low temperature fine-grinder to make red ginseng fine powders.

Then, 50 ml of mixed liquid including β-Glucosidase enzyme in the Preparation Example 2 was mixed with 20 g of finely particulated red ginseng powders and subjected to reaction for 2 hours under a pressure of 2 bar. In this case, in order to increase the contacting frequency between the enzyme and the red ginseng particles and promote the reaction, the enzyme and the red ginseng particles were agitated at 10 rpm.

The red ginseng powders separated after the above process was inoculated with the M. purpureus obtained in Preparation Example 1 and fermented at 30° C. for 3 days. After the fermentation process was finished, purified water was partly mixed to facilitate feeding, and the red ginseng powders were homogenized for 3 times at a rotation speed of 10,000 rpm under a pressure of 300 bar with the homogenizer (HF-93, SMT company, Japan).

The fermented red ginseng powder dispersion completed was sterilized at 70° C. and was completed as a final sample.

### EXAMPLE 2

The red ginseng powder dispersion was prepared with the same method as the Example 1 except that the far infrared ray was radiated for 2 hours and was subjected to heat treatment before grinding red ginseng using a low temperature fine-grinder.

### EXAMPLE 3

The red ginseng powder dispersion was prepared with the same method as the Example 1. However, instead of mixing 50 ml of mixed liquid including β-Glucosidase in Preparation Example 2, 20 ml of mixed liquid including β-Glucosidase in Preparation Example 2 was mixedly used with 30 ml of red ginseng extracts prepared by water extraction.

### COMPARATIVE EXAMPLE 1

The red ginseng powder dispersion was prepared with the same method as Example 1. However, the red ginseng was not ground at a particle size of 10 μm, but was coarsely ground at a particle size of 120 μm to prepare the red ginseng powder dispersion.

### COMPARATIVE EXAMPLE 2

The red ginseng powder dispersion was prepared with the same method as Example 1. However, M. purpureus was not used for fermentation, but Xanthan gum was added to improve the dispersion stability of fine particles.

### COMPARATIVE EXAMPLE 3

The red ginseng powder dispersion was prepared with the same method as Example 1. However, instead of 50 ml of mixed liquid including β-Glucosidase in Preparation Example 2, 50 ml of purified water was used.

### EXPERIMENTAL EXAMPLE 1

### Comparison of Chemical Properties of Red Ginseng Powders

In order to confirm the dispersion stability of red ginseng powders prepared, the chemical properties of the red ginseng powders used in Examples 1 and 2, and Comparative Examples 1 to 3 were compared.

That is, Example 1 uses fine powders to which an enzyme reaction and a fermentation process are continuously applied, and Example 2 performs a far infrared ray heat treatment before grinding the red ginseng.

In comparison, Comparative Example 1 relates to the coarsely-ground red ginseng, Comparative Example 2 relates to the red ginseng which did not go through the fermentation process by the M. purpureus, and Comparative Example 3 relates to the red ginseng which was not treated with β-Glucosidase enzyme.

The water absorption index (WAI), swelling power (SW) and water solubility index (WSI) of red ginseng powders in the Examples 1 and 2, and Comparative Example 1 were measured, and a result thereof is shown in Table 1 below.

TABLE 1

| | WAI | SW | WSI |
|---|---|---|---|
| Example 1 | 2.52 ± 0.21 | 5.25 ± 0.21 | 71.19 ± 0.21 |
| Example 2 | 2.48 ± 0.21 | 5.19 ± 0.21 | 73.52 ± 0.21 |
| Comparative Example 1 | 4.32 ± 0.21 | 9.32 ± 0.21 | 21.37 ± 0.21 |
| Comparative Example 2 | 3.67 ± 0.21 | 8.44 ± 0.21 | 33.19 ± 0.21 |
| Comparative Example 3 | 3.32 ± 0.21 | 8.13 ± 0.21 | 39.32 ± 0.21 |

As a result of comparing the red ginseng powders in Examples 1 and 2, and Comparative Examples 1 to 3 in terms of solubility, the red ginseng particles (Examples 1 and 2) subjected to the β-Glucosidase enzyme reaction and fermented by the M. purpureus have reduced water absorption index and swelling power, and increased water solubility.

In comparison, it was analyzed that the coarsely-ground red ginseng powders, or the red ginseng powders not treated with enzyme or fermentation have relatively low water solubility, so they are not easily dispersed in the dispersion medium.

The water absorption index and swelling power are associated with a porous matrix structure formed by a polysaccharide chain. When the size of the red ginseng particle is large, the matrix structure is extended, and thus water absorption index and swelling power of the powder could increase. Thus, the coarsely-ground powder which has a large particle size and an uneven particle distribution can be easily aggregated. Additionally, since the finely ground powder has a small particle size and an even distribution, the powder cannot easily absorb external moisture, and thus storability and dispersion stability could increase.

Especially, since the inner matrix structure could be denser and the particle shape could become uniform by the β-Glucosidase enzyme reaction and the fermentation process by M. purpureus, the dispersion stability could increase remarkably.

### EXPERIMENTAL EXAMPLE 2

### Evaluation of Functionality on Red Ginseng Powder Dispersion

An evaluation was made for 100 people as to the functionality of red ginseng powder dispersion prepared according to the Examples and Comparative Examples. To this end, the flavor, taste, texture of food, sense of taste and entire satisfaction of red ginseng were investigated. The evaluation included 5 grades such as 5: very excellent/4: excellent/3: good/2: bad/1: very bad. Also, an average was calculated for the evaluation result of the 100 people.

For the experiment, the red ginseng powder dispersion was prepared according to the Examples and Comparative Examples. After the 100 people tasted a small amount of separated sample, they evaluated each item, and a result thereof is shown in Table 2 below. Especially, evaluation on the sense of taste was observed by leaving the prepared red ginseng powder dispersion alone for 8 hours to confirm whether an external layer separation occurs.

TABLE 2

| | Taste | Fragrance of | Texture | Sense | |
|---|---|---|---|---|---|
| | (flavor) | red ginseng | of food | of taste | Assessment |
| Example 1 | 4.3 | 4.2 | 4.3 | 4.5 | 4.2 |
| Example 2 | 4.4 | 4.5 | 4.3 | 4.6 | 4.3 |
| Example 3 | 4.5 | 4.8 | 4.3 | 4.4 | 4.4 |
| Comparative | 2.6 | 2.5 | 2.7 | 2.3 | 2.5 |
| Example 1 | | | | | |
| Comparative | 3.3 | 3.1 | 3.2 | 2.9 | 3.3 |
| Example 2 | | | | | |
| Comparative | 3.2 | 2.9 | 3.1 | 3.5 | 3.1 |
| Example 3 | | | | | |

As shown in the Table 2, the red ginseng powder dispersion to which the β-Glucosidase enzyme reaction and the fermentation by the M. purpureus are continuously applied has excellent texture of food, fragrance, and flavor of red ginseng, and thus satisfaction thereof was high. Additionally, even if the red ginseng was left alone for a long time, layer separation was not observed. Thus, it was evaluated that the red ginseng powder dispersion has excellent product efficiency.

Especially, it was analyzed that the red ginseng powder dispersion in Example 2 in which the red ginseng extracts are mixed has no layer separation, and has remarkably excellent dispersion stability.

### EXPERIMENTAL EXAMPLE 3

### Comparison in Content of Active Ingredients of the Red Ginseng Powder Dispersion

10 ml of the red ginseng powder dispersion prepared in the Examples 1 to 3 and Comparative Examples 1 to 3 was separated as a sample, and the content of active ingredients contained in the sample was analyzed and a result thereof is shown in Table 3 below.

TABLE 3
[Content (mg/100 mL)]

| | | | | Compar- | Compar- | Compar- |
|---|---|---|---|---|---|---|
| | Exam- | Exam- | Exam- | ative | ative | ative |
| | ple 1 | ple 2 | ple 3 | Example 1 | Example 2 | Example 3 |
| Rb1 | 35.19 | 37.21 | 36.21 | 23.21 | 25.62 | 27.15 |
| Rb2 | 41.34 | 43.15 | 45.25 | 21.42 | 23.21 | 29.83 |
| Rc | 27.16 | 28.59 | 27.36 | 20.84 | 24.54 | 28.51 |
| Rd | 17.23 | 20.43 | 21.17 | 11.11 | 12.14 | 16.27 |
| Re | 35.52 | 34.85 | 33.52 | 20.67 | 26.51 | 28.56 |
| Rf | 24.12 | 27.13 | 25.93 | 22.32 | 25.17 | 27.54 |
| Rg1 | 47.11 | 48.51 | 51.25 | 21.61 | 27.53 | 32.64 |
| Rd | 32.53 | 33.34 | 35.87 | 20.12 | 20.53 | 25.61 |
| Rg3 | 24.95 | 26.15 | 29.15 | 20.24 | 20.31 | 27.23 |
| Rg2 | 15.23 | 16.18 | 18.36 | 20.31 | 20.29 | 26.53 |
| Rh1 | 17.11 | 18.28 | 21.71 | 20.17 | 20.24 | 23.24 |
| Rh2 | 28.19 | 29.14 | 32.37 | 10.05 | 10.09 | 14.64 |
| F1 | 9.52 | 10.12 | 15.47 | 10.08 | 10.13 | 11.13 |
| F2 | 13.12 | 14.61 | 17.13 | 10.16 | 10.21 | 11.35 |
| Com- | 6.12 | 6.62 | 8.13 | 10.31 | 10.34 | 11.21 |
| pound | | | | | | |
| K | | | | | | |

Specifically, as shown in the Table 3, Comparative Example 3 which omits the heat treatment process and the process of introducing β-Glucosidase and performs fermentation, and Comparative Example 2 which omits the fermentation process have increased contents of major saponin (Rb1, Rb2, Rc, Rd, Re, Rf, Rg1) and minor saponin (Rd, Rg3, Rg2, Rh1, Rh2, F1). However, it was analyzed that the comparative examples 2 and 3 have lower ginsenoside contents than Examples 1 to 3 according to the present invention.

Especially, in the Examples 1 to 3, the content of ginsenoside such as Rh2, Rg3 and compound K as well as the content of ginsenoside metabolite which is in a form to be easily absorbed increased remarkably. Thus, it is deemed that the red ginseng powder dispersion according to the Examples 1 to 3 will be easily absorbed into the body and the effects thereof on health promotion will be remarkable.

That is, the ginsenoside content is remarkably high in the fermented red ginseng according to the Examples 1 and 2, and thus the fermented red ginseng has remarkable functionality. Additionally, the fermented red ginseng has excellent taste and fragrance compared to the original red ginseng extracts, so it may satisfy the preference of the consumers as high-quality ginseng.

Although the exemplary embodiments of the present invention have been disclosed for illustrative purposes, those skilled in the art will appreciate that various modifications, additions and substitutions are possible, without departing from the scope and spirit of the present invention as disclosed in the accompanying claims. Therefore, it should be understood that the forgoing description is by way of example only, and is not intended to limit the present invention. For example, each constituent explained in singular form may be carried out being dispersed, and likewise, constituents explained as being dispersed may be carried out in combined forms.

The scope of the present invention is defined by the foregoing claims, and it is intended that the present invention covers the modifications or variations of the present invention provided they come within the scope of the appended claims and their equivalents.

1. A method for preparing a fermented red ginseng powder dispersion, comprising:
(a) grinding red ginseng to obtain powdered red ginseng;
(b) introducing the powdered red ginseng into a mixed liquid comprising β-Glucosidase to react with the powdered red ginseng;
(c) inoculating the mixed liquid with a fermented strain for fermentation; and
(d) controlling a concentration of the mixed liquid to a predetermined range.

2. The method of claim 1, further comprising:
(e) homogenizing with a homogenizer; and
(f) performing a low temperature concentration under a vacuum state of 40° C. or below.

3. The method of claim 1, wherein the powdered red ginseng is grinded to a particle size of 2 to 20 μm in step (a).

4. The method of claim 1, wherein the grinding in step (a) is performed under a temperature of −20 to −5° C.

5. The method of claim 4, wherein the powdered red ginseng is grinded to a particle size of 2 to 20 μm in step (a).

6. The method of claim 1, wherein step (b) is performed under a pressure of 1.5 to 4 bar for 1 to 5 hours.

7. The method of claim 1, wherein step (b) is performed at a temperature of 30 to 45° C.

8. The method of claim 1, wherein the mixed liquid further comprises red ginseng extracts.

9. The method of claim 8, wherein the red ginseng extracts are prepared with desalted deep ocean water as a solvent.

10. The method of claim 1, wherein the fermented strain is at least one strain selected from a group consisting of Monascus sp., Lactobacillus sp., Bifidobacterium sp., Prevotella sp., Fusobacterium sp., and Eubacterium sp.

11. The method of claim 10, wherein the strain of Monascus sp. is at least one strain selected from a group consisting of Monascus anka, Monascus purpureus, Monascus pilosus, Monascus ruber, Monascus kaoliang, and Monascus kaling.

12. The method of claim 1, wherein step (c) is performed at a temperature of 25 to 35° C. for 3 to 15 days.

13. The method of claim 1, further comprising performing a heat treatment on the red ginseng before step (a).

14. The method of claim 13, wherein the heat treatment supplies moisture to a surface of the red ginseng and radiates far infrared rays at the red ginseng.

15. The method of claim 13, wherein the heat treatment is performed for 1 to 4 hours.

16. The method of claim 2, wherein step (e) is performed under a pressure of 200 to 800 bar for more than 3 times.

17. The method of claim 2, wherein step (f) is performed under a vacuum state of a pressure of 1 to 20 kPa.

18. A method for preparing red ginseng nanopowder, comprising:
(a) coarsely grinding red ginseng into a particle size of 90 to 150 μm;
(b) radiating far infrared rays at the red ginseng;
(c) finely grinding the red ginseng into a particle size of 2 to 20 μm;
(d) adding β-Glucosidase to the finely ground red ginseng for reaction; and
(e) inoculating a fermented strain for fermentation.

19. The method of claim 18, wherein step (a) is performed by a low speed rotation at 100 to 500 rpm.

20. The method of claim 18, wherein the far infrared rays in step (b) has a wavelength of 20 to 40 μm.

21. The method of claim 18, wherein step (c) further comprises forming a vortex and inducing a collision among particles of red ginseng to finely grind the red ginseng.

22. The method of claim 21, wherein (c) further comprises radiating ultrasonic waves to increase a collision frequency among the particles of red ginseng.

23. The method of claim 22, wherein the ultrasonic waves in step (c) have a vibration frequency of 15 to 20 KHz and an amplitude of 5 to 50 μm.

24. The method of claim 21, wherein step (c) is performed at a temperature of −20 to −5° C.

25. An apparatus for preparing red ginseng nanopowder, comprising:
a first grinding part comprising at least one spiral roller configured to rotate;
a heat treating part configured to radiate far infrared rays;
a second grinding part configured to form vortex and configured to induce collision among particles of red ginseng to finely grind the red ginseng; and
a separating part configured to capture the particles of red ginseng discharged from the second grinding part.

26. The apparatus of claim 25, wherein the spiral roller is configured to rotate at a speed of 100 to 500 rpm.

27. The apparatus of claim 25, wherein the heat treating part is configured to radiate far infrared rays with a wavelength of 20 to 40 μm.

28. The apparatus of claim 25, wherein the second grinding part is configured to increase a collision frequency among the particles of red ginseng by radiating ultrasonic waves.

29. The apparatus of claim 28, wherein the ultrasonic waves have a vibration frequency of 15 to 20 KHz and an amplitude of 5 to 50 μm.

30. The apparatus of claim 25, wherein the second grinding part comprises at least one cooling apparatus configured to supply a low temperature air.

31. The apparatus of claim 30, wherein the low temperature air has a temperature of −20 to −5° C.

32. The apparatus of claim 25, wherein the separating part comprises a vacuum pump configured to decrease an inner pressure.
